(19) United States
(12) Patent Application Publication
(10) Pub. No.: US 2023/0057929 A1
(43) Pub. Date: Feb. 23, 2023

(54) STENT

(71) Applicants: UNIVERSIDAD PONTIFICIA BOLIVARIANA, Medellín (CO); CENTRO CARDIOVASCULAR COLOMBIANO CLÍNICA SANTA MARÍA, Medellin (CO)

(72) Inventors: Liliana María AGUDELO GÓMEZ, Medellín (CO); Andrés Felipe BETANCUR LOPERA, Medellín (CO); Gabriel Jaime COLMENARES ROLDÁN, Medellín (CO); Lina Marcela HOYOS PALACIO, Medellín (CO)

(21) Appl. No.: 17/760,087

(22) PCT Filed: Feb. 3, 2021
(86) PCT No.: PCT/IB2021/050884

§ 371 Date: Oct. 4, 2022

(30) Foreign Application Priority Data
Feb. 3, 2020 (CO) NC2020/0001235

(51) Int. Cl. A61L 31/16; A61L 31/04; A61L 31/10; A61K 31/407; A61K 31/475; A61K 31/337; A61K 31/12

(52) U.S. Cl. CPC A61L 31/16; A61L 31/041; A61L 31/10; A61K 31/407; A61K 31/475; A61K 31/337; A61K 31/12; A61L 2400/12; A61L 2300/416

(57) ABSTRACT

The present disclosure relates to several embodiments of a stent. For example, the present disclosure describes a stent comprising a material selected from a biocompatible material, a bioabsorbable material, and combinations thereof; and particles selected from biocompatible amorphous particles, bioabsorbable amorphous particles, and combinations thereof.

The stent may also include a coating of a material selected from a biocompatible material, a bioabsorbable material, and combinations thereof; nanocapsules and a therapeutic agent encapsulated in the nanocapsules.

The stent disclosed herein enables the walls of an airway or blood vessel to be supported, while there is controlled delivery of the therapeutic agent to said airway or blood vessel to prevent, cure, alleviate or repair symptoms of disease.

### RELATED TECHNICAL FIELD

The present disclosure relates to biocompatible and bioabsorbable medical implants.

More specifically, the present disclosure relates to stents used to prevent the collapse of organic passageways (such as airways and veins) and the dispensing of drugs therein.

### PRIOR ART DESCRIPTION

Stents are devices used to prevent the collapse of organic body conduits such as veins or airways. These stents can be developed in different shapes, sizes, and materials that give the device additional functionality other than preventing the collapse of a body line. The materials that are mainly used in these devices are metals and polymers, whether biodegradable or not. The structure of the stents can be tubes with solid or mesh-shaped walls, and they can also have bifurcations according to the shape of the vein, conduit or pathway where they are housed.

For example, stents used in the airways are developed primarily to prevent death from suffocation caused by deterioration of the tracheobronchial tree due to disease such as cancer, while coronary stents help correct the narrowing of arteries that prevents blood from flowing into the coronary arteries of the heart or veins in other regions of the body.

The prior art teaches stents such as those disclosed in documents US2014/0336747 and US2013/0261735.

Document US20140336747 discloses a method and implant for treating insufficient blood flow to a heart muscle. The implant comprises a hollow elongated body made of a bioresorbable polymer and a bioresorbable sponge, containing an antithrombotic or anticoagulant active agent and a growth factor active agent that promotes angiogenesis and the growth of new capillaries in the heart muscle. The implant disclosed in this document can be in the form of a cylindrical mesh, a single tube or two coaxial internal and external tubes which can be separately prepared and assembled. On the other hand, the bioresorbable material can be a polymer based on polylactide (PLA), polycaprolactone, poly(lactic-co-glycolic) acid (PLGA), polydioxanone, polytrimethylene, among others. Additionally, the strength and stiffness of the implant material can be increased by incorporating reinforcing fillers which may include microcrystalline cellulose, biocrystal, hydroxyapatite, calcium phosphate, zinc, iron, magnesium, and ferric oxide.

Furthermore, this document discloses that the inner tube layer may be made of a high strength bioresorbable polymer, arranged to provide mechanical support. Wherein the inner tube layer may be a magnesium-based bioerodible metal. The outer tube can be made of bioresorbable polymers with a Young's modulus lower than the polymers of the inner layer or a mixture thereof, with active agents so that they are released in a controlled manner to prevent thrombotic closure and promote vascularization.

On the other hand, document US 2013/0261735 discloses a stent composed of an alloy with between 30% w/w and 80% w/w magnesium and a zinc or iron content of 10% w/w to 70% w/w. In some embodiments, the stent comprises a support structure composed of a magnesium alloy and particles, and a coating of bioglass, a biodegradable polymer, and a drug. Said biodegradable polymer may be a biodegradable aliphatic polyester, or may include poly(L-lactide), poly(D-lactide), poly(D,L-lactide), (PLGA), polycaprolactone, poly(trimethylene carbonate), poly(hydroxyalkanoate), poly(butylene succinate), poly (HTH adipate), poly(DTH adipate), poly(DTB adipate), among others. The drug, on the other hand, can be an antiproliferative agent, an anti-inflammatory agent, anticoagulant agents, antithrombotic agents, antimitotic agents, antibiotic agents, antiallergic agents or antioxidant agents.

In addition, US 2013/0261735 also discloses a stent that includes on its surface a barrier layer of a magnesium salt with a solubility product constant of an order of magnitude between 10⁻¹¹ to 10⁻²⁵, said layer can be on all or part of the stent. Some examples of such magnesium salts include magnesium hydroxide (Mg (OH)₂), magnesium ammonium phosphate (MgNH₄PO₄), or magnesium phosphate.

Although the cited documents teach stents, the currently available stents are an incomplete solution for malignant or benign airway obstructions, they present different difficulties depending on whether they are balloon-expandable metal stents, self-expandable metal stents, self-expandable metal stents with coatings, and silicone stents.

Some of these problems include migration or movement of the stent inside the organ conduit, stent fracture due to inadequate mechanical properties of the material, formation of granulation tissue in the organ conduit, and difficulty in removing the stent when it is no longer required, because it becomes embedded in the conduit tissue where it lodges.

Specifically, balloon-expandable metal stents, self-expanding metal stents, or coated self-expanding metal stents are often only temporarily effective for tracheobronchial stenosis, because intraluminal tumor growth or granulation tissue may occur between the stent wires. In addition, these metallic stents are considered permanent because their removal is difficult and may cause serious damage to the organ conduit. In cases of covered stents, the mucosa may degrade the polyurethane membranes. On the other hand, these stents may present displacement due to progressive migration, and their repositioning is not possible

On the other hand, silicone stents have the drawback of requiring rigid bronchoscopy and prior dilation of the organ conduit for their placement, and they are also poorly tolerated in the subglottis. Additionally, these stents limit the diameter available in the organ conduit due to the thickness of their walls, causing occlusion by secretions.

### BRIEF DESCRIPTION OF THE DISCLOSURE

The present disclosure describes embodiments of a stent, particularly a stent comprising a material selected from a biocompatible and/or bioabsorbable material; and particles selected from biocompatible amorphous particles, bioabsorbable amorphous particles, and combinations thereof.

In any of its embodiments, the stent material can be glycolic acid (C₂H₄O₃), lactic acid (C₃H₆O₃), lactic-co-glycolic acid (PLGA), polylactic-co-glycolic acid, polycaprolactone (PLC), chitosan, or combinations thereof. On the other hand, the particles can be amorphous magnesium phosphate, amorphous calcium and magnesium phosphate, hydroxyapatite, magnesium hydroxide, magnesium oxide or combinations thereof.

Also, in any of its embodiments, the stent may include a coating of a material selected from a biocompatible material, a bioabsorbable material, and combinations thereof, nanocapsules, and a therapeutic agent encapsulated in the nanocapsules.

The material of said coating can be cisplatin, collagen, polyvinyl alcohol (PVA), polylactic acid (PLA), polyglycolic acid (PGA), polylactic-co-glycolic acid (PLGA) or combinations thereof. On the other hand, the nanocapsules may be made from glycolic acid, lactic acid, co-glycolic acid, lactic-co-glycolic acid, polylactic-co-glycolic acid, polycaprolactone, chitosan or combinations thereof.

On the other hand, the therapeutic agent encapsulated in the nanocapsules can be selected from the group including antiproliferative agents, antithrombotic agents, anticoagulant agents, anti-inflammatory agents, antineoplastic agents, antiplatelet agents, antifibrosis agents, antimitotic agents, antibiotic agents, antiallergic agents, antioxidant agents, chemotherapeutic agents, cytostatic agents, cell migration inhibitors, immunosuppressants, or combinations thereof.

The stent of the present disclosure can be placed in an organ conduit for the treatment of neoplastic or infectious diseases affecting said conduits. More specifically, the stent can be used for the treatment of infectious diseases of the respiratory system, lung cancer, airway obstruction caused by malignant or non-malignant pathologies, among other diseases.

Furthermore, the stent of the present invention can be placed in a conduit of the circulatory system, such as in a vein or an artery for the treatment of diseases such as arteriosclerosis, cancer or coronary heart disease.

### BRIEF DESCRIPTION OF THE DRAWINGS

FIG. 1 is an isometric view of one embodiment of the stent in the present disclosure.

FIG. 2 is a chart of the roughness of possible particles used as stiffeners in the stent of the present disclosure.

FIG. 3 is a detailed isometric view of one embodiment of the stent in the present disclosure, having an outer coating.

FIG. 4 are views of different nanocapsular structures of an embodiment of the stent in the present disclosure. FIG. 4A illustrates an embodiment of a nanocapsule, where the polymeric membrane contains the therapeutic agent in a liquid core as a molecular dispersion. FIG. 4B illustrates one embodiment of a nanocapsule, where the polymeric membrane surrounds a solid polymeric matrix containing the therapeutic agent. Likewise, FIG. 4C illustrates an embodiment of a nanocapsule that lodges the therapeutic agent both on its surface and in the polymeric membrane.

FIG. 5 is an image of fibers used to coat one embodiment of the stent in the present disclosure, captured by a scanning electron microscope (Scanning Electron Microscope, SEM) at 5000× magnification. These fibers were manufactured from a 13% w/v PVA solution, where PLGA nanocapsules loaded with paclitaxel as a therapeutic agent were embedded.

FIG. 6 is a 5000×SEM image of fibers made from a 13% w/v PVA solution with paclitaxel-loaded PLGA nanocapsules and cross-linked with a 25% glutaraldehyde solution of one embodiment of the stent in the present disclosure.

FIG. 7 illustrates an embodiment of the stent in the present disclosure, set at the bifurcation of an airway.

### DETAILED DESCRIPTION OF THE EMBODIMENTS

Stent

In the present disclosure, a stent (1) comprising a particulate material is described. Particularly, the stent (1) comprises a material (10) selected from a biocompatible material, a bioabsorbable material, or mixtures thereof; and particles (11) selected from biocompatible amorphous particles, bioabsorbable amorphous particles or mixtures thereof.

One of the purposes of the stent is to prevent an organ conduit (such as a vein, airway, or artery) from collapsing. The diameter of the stent, with or without coating, has dimensions that allow it to adapt to the approximate diameters and lengths of the organ conduit where it is located, e.g., if the stent is installed in respiratory conduits, it will have the diameter and length of the branches of the human tracheobronchial tree.

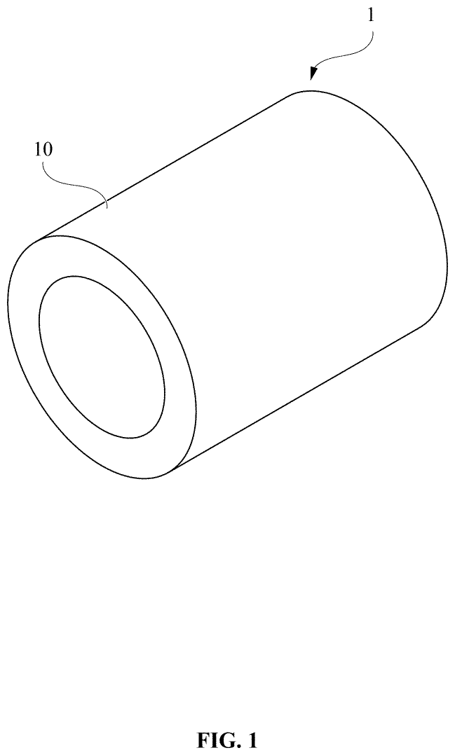

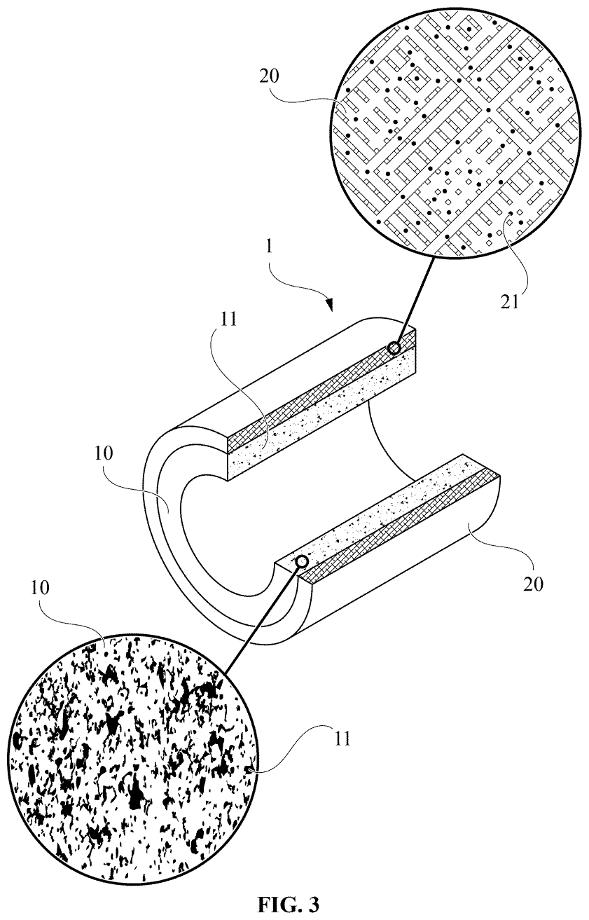

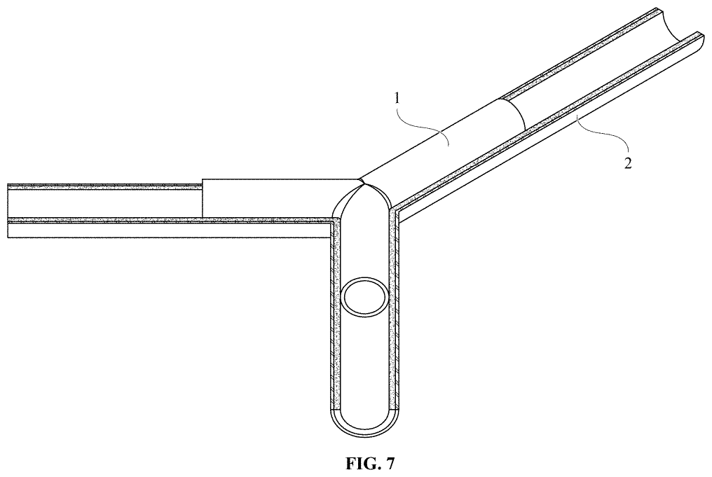

Therefore, the stent can have, e.g., a diameter from 0.4 mm to 0.6 mm, from 0.6 mm to 13 mm, or between 13 mm and 25 mm. Likewise, the stent can have a length of 0.05 cm to 0.17 cm, from 0.17 cm to 5 cm, or between 5 cm and 15 cm. In addition, its shape can be cylindrical as shown in FIG. 1 and FIG. 3 or branched as illustrated in FIG. 7. However, the dimensions of the stent will depend on the conduit size and shape where the stent is placed.

One of the objectives of including particles (11) in the stent (1) of the present disclosure is to improve its mechanical properties. By including amorphous particles (11) in the stent (1) material (10), both the average Young's modulus and the material hardness can be increased by more than 50% and 100%, respectively, relative to the material without particles. The foregoing shows that the mechanical resistance of the stent (1) is increased, preventing the collapse of the conduit where the stent (1) is located. Particularly, inflammatory and proliferative diseases of the airways cause such collapse, which can be prevented with the use of the stent (1) in the present disclosure.

Additionally, including amorphous particles (11) in the stent (1) material (10) allows the mechanical properties of said stent (1) to be maintained for a period of time from 3 to 36 months. In one embodiment of the disclosure, the amorphous morphology of the particles (11) increases the bioabsorbability of the stent (1), in such a way that it is assimilated by the biological tissue in a time between 6 and 18 months, which is advantageous for certain applications where intermediate periods of stent (1) permanence within an organ conduit are required. The amorphous particles (11) are more quickly assimilated by a biological organism, compared to the particles of crystalline morphology.

For example, one of the advantages provided by the addition of amorphous magnesium phosphate particles (11) is that they prevent the calcification of the organic tissue surrounding the stent (1), preventing its stiffening.

In particular, the present disclosure describes a stent (1) comprising between 51% w/w to 60% w/w, or between 61% w/w to 80% w/w or between 81% w/w to 99% w/w of a material (10) that is selected from a biocompatible material, a bioabsorbable material, and combinations thereof. The stent (1) also comprises between 40% w/w and 49% w/w, or between 20% w/w and 39% w/w, or between 1% w/w and 19% w/w of selected particles (11) among biocompatible amorphous particles, bioabsorbable amorphous particles and combinations thereof. An embodiment of said stent (1) can be seen in FIG. 1 and FIG. 3.

In some of the embodiments, the stent can include polylactic-co-glycolic acid PLGA 8515 or PLGA 5050 and amorphous magnesium phosphate (AMP) particles, as material (10). In addition, said stent may include a polyvinyl alcohol coating with polylactic-co-glycolic acid nanocapsules containing Paclitaxel or Curcumin as therapeutic agents.

Stent Material

The stent (1) material (10) is a biologically inert polymeric matrix to be incorporated into a living organism and replace or restore some function thereof, remaining in permanent or intermittent contact with body fluids without deteriorating or interacting with the living system at issue. In particular, the material (10) from which the stent is made is selected from a biocompatible material, a bioabsorbable material, and mixtures thereof, preferably it is a biocompatible and bioabsorbable material or a mixture of biocompatible and bioabsorbable material.

For interpretation purposes of the present invention, biocompatible materials will be understood as materials accepted for use in biological tissues, which preferably comply with ISO-10993 standard: “Biological Evaluation of Medical Devices.” Some materials are for example: Polyaryletherketone (PAEK), Polyetheretherketone (PEEK), High Density Polyethylene (HDPE), Ultra High Molecular Weight Polyethylene (UHMWPE), Polymethyl Methacrylate (PMMA), Commercially Pure Titanium (ASTM F67), Titanium Alloys (ASTM B265) and AISI 316L Stainless Steel, collagen, silicone, glycolic acid, polyglycolic acid, lactic acid, polydioxanone, polycaprolactone, chitosan, lactic-co-glycolic acid, polylactic-co-glycolic acid, co-polymers thereof and mixtures thereof.

On the other hand, bioabsorbable materials are those in which their substance or the products of their decomposition pass through or are assimilated by cells or tissue over time (ISO-10993 standard: “Biological Evaluation of Medical Devices”). Said bioabsorbable materials can simultaneously be biocompatible, e.g., polyglycolic acid (C₂H₂O₂)—, polylactic acid (C₃H₄O₂)ₙ, polydioxanone, polycaprolactone (PCL) and polylactic-co-glycolic acid.

More particularly, the biocompatible and/or bioabsorbable materials of the stent (1) can be selected from the group consisting of collagen, silicone, glycolic acid (C₂H₄O₃), polyglycolic acid (C₂H₂O₂)—, lactic acid (C₃H₆O₃), polydioxanone, polycaprolactone (PLC), chitosan, lactic-co-glycolic acid, polylactic-co-glycolic acid, and mixtures thereof. Each of these materials has advantages and disadvantages, as described below.

Collagen is used in the repair of chemical-mechanical damage or trauma, whether in skin or mucous membranes, due to its biocompatibility and its ability to promote wound healing, its function is mechanical and supportive, being an important component of the extracellular matrix (ECM). In addition, it is approved by the Food and Drug Administration of the United States (FDA). Specifically, type II collagen is suitable for release processes due to its short biodegradability time (3-6 months).

Silicone is an FDA-approved material used for the manufacture of bronchial and cardiac stents, medical devices and coatings. However, stents made of this material generally require the use of anesthesia and a rigid bronchoscope for installation or removal, and tend to migrate more frequently than metal stents. Some of their disadvantages are that silicone stents facilitate the adhesion of secretions, have an unfavorable relationship between wall thickness and diameter compared to metal stents, are difficult to use in irregular pathways and interfere with the mucociliary mechanism and they are not biodegradable.

In order to avoid the mechanical complications associated with nonabsorbable stents, bioabsorbable materials such as polyglycolic acid (PGA), polylactic acid (PLA), polydioxanone (PDO), polycaprolactone (PCL), and poly(lactic-co-glycolic) acid (PLGA) can be used.

Polyglycolic acid (PGA) is generally used for subcutaneous sutures, intracutaneous closures, abdominal and thoracic surgeries. It has been approved by the FDA for clinical uses such as bioabsorbable sutures and bone fixation devices. However, it typically loses its mechanical strength over a period of 2 to 4 weeks after implantation, and biodegrades within 1 to 2 months. It has the advantages of high initial tensile strength, smooth passage through the tissue, easy handling, excellent knotting ability and secure knot tying.

Polylactic acid (PLA) is used in cardiac stents, sutures, bone systems, and cosmetic correction of scars and wrinkles. It has been approved by the FDA for clinical uses such as bioabsorbable sutures and bone fixation devices. Additionally, it is used to restore and/or correct signs of facial fat loss (lipoatrophy) in people with human immunodeficiency virus. However, it may cause delayed appearance of subcutaneous papules, ectropion, skin hypertrophy, injection site abscess, or injection site atrophy. Its biodegradation time is 6 to 9 months.

Polydioxanone (PDO) is a polyester polymer approved by the FDA for use in cardiac stents, coatings, and sutures. It enhances the cohesion of cells at the cardiac level. It is biocompatible, resorbable and its biodegradation time is 6 months. One of the disadvantages it presents is that it facilitates inflammation and hyperplasia in the tissues.

Polycaprolactone (PCL) is made from petroleum derivatives, it is non-toxic and its biodegradation time is 24 to 60 months. It is especially used for the preparation of long-term implantable devices and has been used in applications in the human body as a drug delivery or adhesion barrier. Contraindications are unknown.

Polylactic-co-glycolic acid (PLGA) is a copolymer obtained from polylactic acid (PLA) and polyglycolic acid (PGA). PLGA is generally an acronym for poly D, L-lactic-co-glycolic acid where the D- and L-lactic acid forms are in equal proportion. It is an ideal polymer in drug delivery and in biomedical applications, such as the construction of a stent body.

PLGA can be processed into almost any shape and size and can encapsulate molecules of any size. Additionally, PLGA is soluble in a wide range of common solvents, including chlorinated solvents, tetrahydrofuran, acetone, or ethyl acetate. In water, PLGA biodegrades by hydrolysis of its ester linkages. The presence of methyl side groups in PLA makes it more hydrophobic than PGA and therefore lactide-rich PLGA copolymers are less hydrophilic, absorb less water and subsequently degrade more slowly. Due to PLGA hydrolysis, parameters that are typically considered invariant descriptions of a solid formulation can change over time, such as glass transition temperature (Tg), moisture content, and molecular weight.

The physical properties of PLGA depend on multiple factors, including initial molecular weight, lactide to glycolide ratio, device size, exposure to water (surface shape), and storage temperature. The mechanical strength of PLGA is affected by physical properties such as molecular weight and polydispersity index, these properties are also related to the ability to be formulated as a drug delivery device and can control the rate of device degradation and hydrolysis.

PLGA is classified by the FDA as a substance or mixture that does not contain components considered persistent bioaccumulative and toxic (PBT), very persistent and bioaccumulative (vPvB) at levels of 0.1% or higher. Its acute oral toxicity LD50—rate—>10,000 mg/kg and the International Agency for Research on Carcinogens (IARC) indicates that no component of this product, presenting levels greater than or equal to 0.1%, is identified as a probable human carcinogen.

Additionally, PLGA is suitable for manufacturing the stent (1) of the present invention, because it breaks down into two materials that are part of the body's biological synthesis, such as glycolic acid and lactic acid. PLGA does not present contraindications when used in the airways, such as restenosis, allergies, intolerances and trauma caused by the removal of the device, which usually may be even more harmful.

In one embodiment of the invention, the stent material (10) (1) body is biocompatible and bioabsorbable, approved by the FDA and can be reinforced to improve its mechanical properties.

When the stent material (10) (1) body is made from PLGA, the molar ratio between the lactide and glycolide units of said PLGA copolymer is one of the factors determining its bioabsorption time, i.e., the stent degradation is faster if the number of moles of glycolide units is greater than the number of moles of lactide units, because PGA dissolves easily in water and PLA is less akin thereto. The foregoing is detailed in the following table:

TABLE 1
Degradation time of PLGA according to its molar ratio.

| | Molar Ratio | | Degradation | |
|---|---|---|---|---|
| Material | PLA | PGA | Time | |
| PLGA | 90 to 99 | 1 to 10 | >3 | years |
| PLGA | 60 to 89 | 11 to 40 | 1-2 | years |
| PLGA | 30 to 59 | 41 to 70 | 1-1.5 | years |
| PLGA | 15 to 29 | 71 to 85 | <1 | year |
| PLGA | 1 to 14 | 86 to 99 | <6 | months |

In accordance with the foregoing, if the body, the coating and/or the nanocapsules of the stent are manufactured with PLGA, their degradation time can be predefined as needed, taking into account the molar ratio of their components. Additionally, the inherent viscosity of PLGA also contributes to a more or less slow degradation. Preferably said inherent viscosity for the PLGA used as the stent body material is 1.7d1/g to 7d1/g.

For the understanding of the present invention, it is important to mention that the stent (1) is manufactured from a solution containing the stent (1) material (10) mentioned as a “polymeric matrix” and other components among which particles (11), surfactants, reagents, additives (plasticizers, stabilizers, lubricants, among others) and solvents, are included. Said solution undergoes a curing process to obtain the solid stent (1) body, which has different mechanical and chemical properties according to the composition and manufacturing method used, as will be detailed later.

Particles

As mentioned above, in order to generate the reinforcement and improvement of the mechanical properties, the stent (1) also comprises bioabsorbable and/or biocompatible particles (11). For example, said particles (11) can be selected from the group consisting of calcium hydroxyapatite (HA), calcium amorphous phosphate (CMP), magnesium amorphous phosphate (AMP), magnesium oxide (MgO), magnesium metal, magnesium hydroxide (Mg(OH)₂), and mixtures thereof. Preferably the stent comprises “amorphous” particles, such as calcium amorphous phosphate (CMP) or magnesium amorphous phosphate (AMP). Where amorphous particles are understood as particles with acicular morphology, or in the form of bars.

Furthermore, in one embodiment of the invention the representative sphere diameter of the particles (11) is between 1 nm and 1 μm, or between 1 nm and 10 μm. The rod-shaped particles 11 can have lengths from 1 μm to 30 μm and diameters between 1 nm and 5 μm. In another example, the particles (11) may have a particle size less than the opening size of a 20 μm ASTM sieve.

In one embodiment of the stent (1), the reinforcing particles (11) are amorphous magnesium phosphate (AMP) which is a biocompatible, biodegradable and bioactive material. Therefore, it can be used as bone substitute, filler in biocomposites and coatings which will not only support the sustained release of magnesium and phosphate ions, but also can significantly promote biological activities. AMP is a material that is synthesized using microwave methods, such as microwave-assisted hydrothermal synthesis.

In one embodiment of the stent (1), amorphous magnesium phosphate particles (11) can be used having different shapes and sizes, mainly acicular or rod-shaped particles that help generate significant reinforcement thanks to their elongated shape, with widths ranging from the nanometric scale to several microns and lengths that may exceed up to 10,000 nm.

Stent Body Manufacturing Method (Polymer+Particles)

Regarding the stent manufacturing process, it is important to mention that the particles (11) are added to the polymeric matrix of the stent, when said polymeric matrix is in a liquid state and forms a solution.

On the other hand, the integration of the particles (11) in the stent (1) material (10), such as those described above, can be carried out in different ways. An example of a method for manufacturing an embodiment of the stent (1) includes using the following elements:
Internal diameter tube (d1);
Solid rod of diameter (d2) where d2<d1;
Two caps with first holes configured to enter a prepared solution containing the polymer, surfactants, reagents, additives (plasticizers, stabilizers, lubricants, among others) and solvents and second holes configured to facilitate the release of gases that evaporate from the solution that is introduced, during the curing process of the stent body (1).

This assembly is stipulated so that the constructed stent (1) has a thickness defined by the difference between the diameters of the tube and the rod. It should be noted that the construction measures of the device will depend on the organic conduit (such as a vein, an airway or an artery) where the stent (1) is to be installed.

In an embodiment of the manufacturing method of a stent (1) embodiment, the following steps are executed:
preparing the solution containing the polymeric matrix and the particles (11) as nanoreinforcers;
releasing the agent to the rod and to the inside of the tube in order to avoid damage to the stent (1) at the time of demolding it;
arranging the rod inside the tube and locating the plugs at the ends of the tube to configure a cylindrical-shaped chamber between the tube and the rod;
injecting the solution from the lowest part of the stent (1) upwards through one of the cap holes;
allowing the solvent to evaporate from the solution for an estimated time and then slowly removing the caps, the rod and finally the tube, to avoid damaging the solidified stent (1).

Another alternative to build the stent (1), especially in cases where the diameters are less than 3 mm, is the implementation of Dip Coating. This technique takes advantage of the viscosity and drying speed of a solution to create layers on a cylindrical surface until reaching the desired thickness and size. The cylindrical surface is immersed in the solution to form a coating layer on said cylindrical surface. The entry and extraction speed of the cylindrical surface into the solution is predetermined under controlled temperature and atmospheric conditions, and the number of times the cylindrical surface is immersed will depend on the thickness of the cylinder walls that it is desired to establish. The Dip Coating technique consists of immersing a substrate to be coated, such as a plate, bar, or cylinder, in a liquid and removing the substrate with a predetermined extraction speed under controlled temperature and atmospheric conditions. The thickness of the coating is mainly defined by the extraction speed, the solid content and the liquid viscosity.

The solvent used in the manufacture of the stents (1) is any known to a person of ordinary skill in the art, where the solvent dilutes a solute, the solute being the polymeric matrix.

The solvent must not interfere with the properties of the stent material (10) or polymeric matrix, i.e., it must not leave traces of another compound or affect the bioabsorption, biocompatibility and toxicity of the stent (1). For example, when the polymer matrix is PLGA, a solvent or reagent is used, selected from the group consisting of chloroform (CHCl₃), acetone (C₃H₆O), dichloromethane (CH₂Cl₂), hexafluoroisopropane (CF₃)₂CHOH, acetic acid (CH₃—COO) and mixtures thereof.

Coating

Referring to FIG. 3, in one embodiment of the stent (1), it may include a coating (20). Particularly, the coating comprises a biocompatible and/or bioabsorbable material between 51% w/w to 60% w/w, or between 61% w/w to 80% w/w or between 81% w/w to 99% w/w. The coating in turn comprises nanocapsules (21); and a therapeutic agent encapsulated in the nanocapsules (21). Particularly, the coating also comprises between 40% w/w and 49% w/w, or between 20% w/w and 39% w/w, or between 1% w/w and 19% w/w of nanocapsules (21); and a therapeutic agent encapsulated in the nanocapsules (21).

One of the coating functions is to release the nanocapsules for a predefined time and quantity, in order to dose the therapeutic agent according to its application and the patient needs. One of the coating advantages of the present invention is that it allows a controlled release of the therapeutic agent.

For example, in the treatment of lung cancer, antiproliferative agents ideally continue to be released weeks after the stent (1) is placed in the airway. However, if the antiproliferative agent is dispensed for a long time, tissue necrosis is generated by preventing cells from reproducing.

Accordingly, the coating of the present disclosure allows to obtain, e.g., a therapeutic agent release rate of 32% during the first month. These values may change if the configuration, geometry or materials of the coating are modified.

Coating Material

The biocompatible or bioabsorbable materials of the coating can be selected from the group consisting of cisplatin, collagen, polylactic acid (PLA), polyglycolic acid (PGA), polylactic-co-glycolic acid (PLGA), polyvinyl alcohol (PVA), and mixtures thereof. The polarity of the therapeutic agent and the nanocapsules must be taken into account in the selection of said coating material.

For example, in an embodiment of the invention, where the therapeutic agent is Paclitaxel, the PLA of the coating can react with the PLGA nanocapsules, allowing the dispersion of Paclitaxel. Similarly, in the case of using PLGA as a coating, the PLGA dissolves in the solvents used for the nanocapsules, which are also PLGA. For this reason, in said example, PVA and collagen are more suitable as a coating material.

Type II collagen is bioabsorbable in living tissue, taking into account that bioabsorption could be carried out immediately by the human body, the collagen cross-linking technique improves its hydrophobicity delaying the release time of the nanocapsules.

On the other hand, polyvinyl alcohol (PVA) is a polymer that is obtained by hydrolysis of polyvinyl acetate. It is one of the polymers with the greatest application in the industry as an adhesive, paint, food and medicine. In one embodiment of the invention, PVA is used as the coating material for the stent (1) and contains the nanocapsules (21).

Nanocapsules

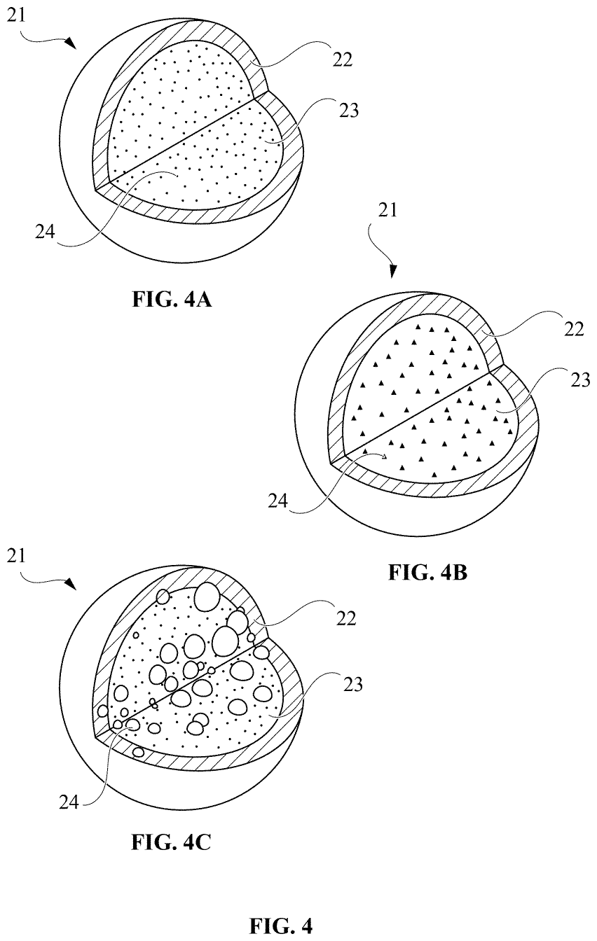

Now, as mentioned above, said coating also comprises nanocapsules (21). Referring to FIG. 4, in relation to the nanocapsules (21), these are defined as nano-vesicular systems that exhibit a typical layer-core structure in which the active ingredient or therapeutic agent (24) is confined within a polymeric membrane (22). The polymeric membrane (22) may contain the therapeutic agent (24) in a liquid core (23) as a molecular dispersion, as seen in FIG. 4A, or it can surround a solid polymeric matrix (23) containing the therapeutic agent (24) as illustrated in FIG. 4B. Likewise, this deposit can be lipophilic or hydrophobic according to the method of preparation and the raw materials used. In addition, taking into account the operational limitations of the preparation methods, the nanocapsules (21) can also carry the therapeutic agent (24) on their surfaces or be embedded in the polymeric membrane (22) as seen in FIG. 4C.

One of the functions of the nanocapsules (21) is to release the therapeutic agent (24) in a controlled manner, achieving the correct amount of the active agent, for a suitable period of time and in the precise place where it needs to be administered. This release method is used to extend the time the therapeutic agent is effectively present using a single dose, and to eliminate or minimize concentrations exceeding therapeutic requirements. The mechanism by which the release of the therapeutic agent occurs is influenced by different factors such as: the material of the nanocapsules (21) (composition, structure, degradability), the medium in which the release occurs (pH, enzymes, ionic strength, temperature) and the type of therapeutic agent (hydrophilicity, stability and interaction with the nanocapsule).

The material of the nanocapsules (21) must be biocompatible or bioabsorbable and must allow the therapeutic agent to be released, for this its inherent viscosity must preferably be less than 2d1/g. The material of the nanocapsules (21) can be selected from the group consisting of glycolic acid (C₂H₄O₃), lactic acid (C₃H₆O₃), lactic-co-glycolic acid (PLGA), polylactic acid-co-glycolic, polycaprolactone (PLC), chitosan and mixtures thereof. In addition, the size of the nanocapsules (21) can be between 1 nm and 1000 nm or the necessary size for them to be in the biocompatible or bioabsorbable material, this size also affects the therapeutic agent dosage.

The nanocapsules (21) and the therapeutic agent may have the same or different polarity. To carry out the encapsulation, if the polarity of the nanocapsules (21) and the therapeutic agent is the same, the latter is added in the same phase of the nanocapsules, if they are of opposite polarity they go in opposite phases, the surfactant is the one in charge of their interaction to perform the encapsulation. For example, it is possible to encapsulate Paclitaxel in PVA or Collagen, which are hydrophilic, and also Paclitaxel in polycaprolactone PCL or in PLGA, which are hydrophobic.

Manufacturing Nanocapsules

The nanocapsules (21) can be manufactured by means of nanoprecipitation, emulsion-diffusion, double emulsification, emulsion coacervation, polymer coating, layer by layer and by means of the combination or variation of the previous methods, or any other method for the manufacture of nanocapsules known to a person of ordinary skill in the art. In one embodiment of the invention the coating has a therapeutic agent concentration of between 50 μg/cm² to 150 μg/cm². The dosage and concentration of the therapeutic agent is associated with its toxicity and therapeutic effect. Very low concentrations do not cause therapeutic effect and very high concentrations may cause adverse toxic effects.

Therapeutic Agent

On the other hand, and in accordance with what was mentioned throughout the description, one of the functions of the coating is to contain the therapeutic agent. Said therapeutic agent is a substance whose function is to prevent, cure, alleviate or repair the symptoms of a disease in an animal organism.

The therapeutic agent can be selected from the group including, but not limited to:

antiproliferative agents, antithrombotic agents, anticoagulant agents, anti-inflammatory agents, antineoplastic agents, antiplatelet agents, antifibrosis agents, antimitotic agents, antibiotic agents, antiallergic agents, antioxidant agents, chemotherapeutic agents, cytostatic agents, cell migration inhibitors, immunosuppressants, and combinations thereof.

Examples of chemotherapeutic agents are Mitomycin, Vincristine, Paclitaxel and Curcumin. Examples of immunosuppressive agents are sirolimus, tacrolimus, and everolimus. Examples of anti-inflammatory agents are dexamethasone and Biolimus A9. Examples of antineoplastic agents are taxanes such as docetaxeland cabazitaxel.

For example, Paclitaxel exhibits antiproliferative activity against different cells, such as vascular smooth muscle cells, fibroblasts, endothelial cells. In addition, it inhibits restenosis in cardiology, vascular surgery and other fields, and its use in the tracheal mucosa may inhibit the formation and development of tracheal granulation tissue.

Paclitaxel is active against a wide range of cancers that are generally considered resistant to conventional chemotherapy, such as ovarian and breast cancer. Furthermore, Paclitaxel can be used for antiproliferative treatment in coronary artery disease and bronchial obstructive disease. However, Paclitaxel can cause allergies to cyclosporine, teniposide, and drugs containing polyoxyethylated castor oil.

Curcumin, e.g., is used for liver and skin protection, in addition to being used to treat diseases such as jaundice and biliary fevers. Curcumin, a diferuloylmethane, exhibits anticancer properties in vivo and has been shown to inhibit different tumor cells present in living beings. Curcumin exerts anticancer effects by inhibiting the proliferation and metastasis of cancer cells and inducing cell cycle arrest and apoptosis.

In one embodiment of the stent (1), the therapeutic agent nanocapsules can be synthesized using a variation of the nanoprecipitation technique. The system uses two or more streams that are mixed through a recirculation system to form polymeric nanoparticles and nanocapsules with different particle size distributions and different amounts of encapsulated active agent that can be designed for fast or slow release. The length of the system can be varied from 30 cm to 15 m and the internal diameter of the reactor can be from 0.79 mm to 38 mm.

The material of the tubular reactor is recommended to be vinyl-methyl-silicone (VMQ silicone) or similar material resistant to the solvents used in the organic phase and that meets Class VI specifications of the United States Pharmacopeial Convention USP (United States Pharmacopeial Convention), which judges the suitability of the plastic material intended to be used as containers or accessories for parenteral preparations.

The size and distribution of the particles can be modulated by changing the internal diameter of the injection needle according to the measurements of the Birmingham system (18 G, 20 G, 21 G, 22 G, 23 G, 25 G, 27 G, 29 G, 30 G, 31 G) and modulating the speed of injection. In order to obtain Curcumin or Paclitaxel loaded nanoparticles, an organic phase solution and an aqueous phase solution can be prepared.

The organic phase containing the polymer, the active agent and one or more organic solvents. On the other hand, the aqueous phase, which is made up of water and a surfactant or more additives (surface modifiers, molecules, functional groups, water-soluble polymers, among others) that help the formation of nanoparticles and also modify the drug release profiles with these.

After carrying out the nanoprecipitation, the solvent must be recovered by vacuum-assisted evaporation and the subsequent recovery or concentration of the nanoparticles must be carried out according to their final application. The nanoparticles can be freeze-dried or dried using the spray dryer technique.

Coating Manufacturing Method and Arrangement on the Stent Body

After the construction of the stent body (1), which was explained above, the coating is applied thereto. In one embodiment of the stent (1), a procedure is used to coat the stent (1) body that includes the following steps:
synthesizing the nanocapsules (21) with therapeutic agent using nanoprecipitation;
synthesizing the polymeric solution of the coating material;
mixing the polymer solution in a suitable solvent plus the therapeutic agent-loaded nanocapsules;
dispensing the polymer solution of the nanocapsule coating material into a syringe;
carry out the electrospinning process on the stent (1) body.

The nanoprecipitation method, also known as “solvent displacement or interfacial deposition,” consists in two phases needed for the formation of the nanocapsule and encapsulation of the therapeutic active ingredient: solvent and non-solvent. Generally, the solvent is an organic medium, while the non-solvent is primarily water. However, it is possible to use two organic phases or two aqueous phases as long as the conditions of solubility, insolubility and miscibility are satisfied.

In relation to electrospinning, this allows a filament with a diameter between 100 nm and 500 nm to be deposited on a plate or around a cylinder. The electrospinning process allows the production of nanofibers with controlled surface area, porosity, orientation, dimensions and mechanical properties.

The electrospinning process is performed by injecting the solution onto the rotating body of the stent, while a voltage of 15 kV to 20 kV is applied to the solution, which is injected at a rate of 0.005m1/h to 1 ml/h at a distance from the stent body (1) from 10 cm to 20 cm.

During electrospinning, the stent (1) body rotates on its own axis at a speed of 5 rpm to 25 rpm, to receive the solution thread evenly on the surface of the stent (1) body. Additionally, the work cabin where the electrospinning is carried out, has a relative humidity of less than 60% and a temperature of 15° C. to 35° C.

Additionally, it is possible to carry out a crosslinking process of the filament obtained by electrospinning seeking greater resistance to water or swelling of the polymer to avoid the rapid release of the therapeutic agent or the rapid degradation of the coating material. For the understanding of the present invention, “swelling” is defined as the hydration process suffered by polymers that are related to water when they come into contact with it. This process is the cause of the increase in volume and the solubilization of the polymer over time.

As for the crosslinking method, this consists of merging the contact areas between the fibers, this is achieved by exposing the fibers of the coating for a period of time to the vapors of a solution, which can be Glutaraldehyde (GLU) at 25%, a 25% glutaraldehyde and 32% w/v hydrochloric acid (HCl) solution in a 3:1 ratio, or a 50% glutaraldehyde and 99% ethanol solution in a 1:1 ratio.

Uses

In one embodiment of the stent (1), it can be used in the treatment of cancer, e.g., kidney cancer, breast cancer, lung cancer, esophageal cancer, colon cancer, pancreatic cancer, colorectal cancer, gastric cancer, kidney cancer metastasis, tracheal cancer, bronchial cancer, among others. One of the functions of therapeutic agents is to counteract the restenotic process, for which immunosuppressive, antiproliferative, anti-inflammatory, antithrombotic and procurative agents are used.

Preferably, the drug should inhibit the formation of neointimal hyperplasia by suppressing one or more of the processes of platelet activation, acute inflammation, smooth muscle cell migration, smooth muscle cell proliferation, extracellular matrix production, angiogenesis, and vascular remodeling. Also, it must preserve vascular healing and allow reendothelialization of the injured vessel wall.

Particularly, in the treatment of cancer, the stent (1) retains its properties between 12 and 18 months, thanks to the fact that amorphous particles (11) are included in the stent (1) material, which is, e.g., PLGA. Said amorphous particles (11) improve the mechanical properties of the stent (1) as explained above.

Another therapeutic agent used in one embodiment of the invention may be Umirolimus, which is a biodegradable polylactic acid polymer that has anti-inflammatory and antiproliferative activity and can reversibly inhibit growth factor-stimulated cell proliferation. However, its use can cause decreased wound healing and thrombocytopenia.

Sirolimus is another therapeutic agent used to combat some cancers, by slowing cell proliferation and tumor growth. It is also used to treat kidney conditions and prevent rejection of transplanted organs, such as the kidney. Also, it is not a calcineurin inhibitor. A possible collateral effect of Sirolimus is to cause a decrease in wound healing and thrombocytopenia, in addition, pulmonary toxicity is an important complication derived from the use of Sirolimus.

On the other hand, Everolimus is an immunosuppressant used for transplantation of organs such as kidney, heart, lung and pancreas and in the treatment of kidney or breast cancer. However, its use can decrease the ability to fight infections caused by bacteria, viruses and fungi, and increase the risk of serious or fatal infection.

Finally, Zotarolimus is used to reduce early inflammation and restenosis in coronary stents, and is not thrombogenic. However, its poor solubility in water prevents rapid release into the circulation.

On the other hand, in several of its embodiments, the stent (1) can be used in a treatment for lung cancer or airway obstruction caused by non-malignant pathologies.

For example, obstruction of the tracheobronchial tree is common among patients diagnosed with lung cancer. These patients may develop dyspnea, stridor, intractable cough, hemorrhage, atelectasis or post-obstructive pneumonia, or a combination of the aforementioned manifestations due to extension of a tumor to the tracheobronchial tree.

These patients are usually non-surgical candidates, due to their physiological and oncological conditions, for which the optimal treatments for patients with central airway obstruction include radiotherapy, laser therapy, photodynamic therapy, cryotherapy and the implantation of airway stent (1), which can be implanted with or without a bronchoscope.

On the other hand, central airway obstructions can also be caused by non-malignant pathologies, such as post-intubation and post-tracheostomy stenosis, followed by foreign bodies and tracheobronchomalacia. Other causes, such as those secondary to infectious processes and systemic diseases (sarcoidosis, amyloidosis, Wegener's disease, relapsing polychondritis, and tracheobronchopathy osteochondroplastic). And finally, idiopathic tracheal stenosis and post-lung transplant bronchial stenosis, which are less frequent.

The stent (1) of the present disclosure meets the features required in a bronchial stent for the treatment of the aforementioned conditions. The stent (1) is biocompatible and bioabsorbable, radiopaque and does not generate an inflammatory reaction. In addition, it can have features similar to those of the airway to reduce the accumulation of secretions, it can be impermeable and prevent the growth of tumors or granulation tissue inside the stent (1), it can be flexible and capable of adapting to the movement of the airways. Additionally, its design can prevent migration from its initial position within the airway and its particles (11) give it a radial resistance allowing it to keep the airway open.

### Example 1—PLGA Stent Reinforced with Amp Particles

Referring to FIG. 3 and FIG. 7, a stent (1) for placement in an airway (2) was developed. Said stent (1) had a body made with PLGA as a polymeric matrix and amorphous magnesium phosphate (AMP) particles as a reinforcer, using the Dip Coating technique.

The construction of the stent (1) began with the integration of the AMP in the PLGA. This process was carried out by adding between 1% and 5% by weight of AMP with respect to the weight of PLGA. Between 12 and 16 mL of CHCl₃ was taken and the weight percentage of AMP was added with respect to 1 g of PLGA to be prepared. This solution with the nanoparticles is sonicated for 10 minutes without adding the PLGA.

After the homogenization of the solution with AMP, portions of 0.1 g of PLGA were added little by little until completing 1 g, between each addition of PLGA it was ensured that the previous addition was completely dissolved to add the following one, in order to guarantee the complete homogenization and dissolution of the PLGA in all the material. In this range of addition, the AMP gave the stent structure (1) greater mechanical resistance, by increasing the average Young's modulus by more than 50%, hardness by more than 100%, in addition to reducing roughness by more than 40% compared to PLGA without any reinforcement.

After integrating these two components (PLGA-AMP), the stent body was constructed (1). This process was carried out using Dip Coating, a technique that takes advantage of the viscosity and rapid drying of the PLGA/AMP solution to create layers on a cylindrical surface until the desired thickness and size are reached. The cylindrical surface is immersed in the PLGA/AMP solution to form a coating layer of PLGA/AMP on said cylindrical surface. The rate of entry of the cylindrical surface into the solution was between 4 mm to 8 mm per second, and the number of times the cylindrical base is immersed will depend on the thickness of the cylinder walls to be established.

### Example 2—PLGA Stent Reinforced with AMP Particles, Coated with PVA with Nanocapsules Containing Paclitaxel and Curcumin

A stent (1) like the one in Example 1 was developed and coated using the electrospinning technique, taking PVA as the coating matrix and PLGA nanocapsules containing Paclitaxel and Curcumin as antiproliferative therapeutic agents for cancer.

Prior to the stent body coating process (1), PLGA nanocapsules with Paclitaxel and Curcumin were prepared by means of a process based on the nanoprecipitation technique.

After obtaining the nanocapsules with Paclitaxel and Curcumin, a 10% PVA solution was prepared and 3% by weight of the nanocapsules were added, which was then used to generate the stent coating (1) using electrospinning.

The equipment used for electrospinning had a syringe pump with which the system feed flow could be varied, a high voltage source operating at 15 KV, which generated a potential difference that allowed the fibers to form continuously from the tip of the syringe to the manifold. The equipment had a manifold in the form of a circular drum that rotated at 10 rpm and was located at a distance of 17 cm from the tip of the syringe. The polymer solution in a solvent plus the nanocapsules loaded with the therapeutic agent were placed in 5 to 10 mL disposable syringes and the feed flow was 0.3 mL/h.

Additionally, the work cabin where the electrospinning was carried out was brought to a relative humidity below 60% and a temperature of 28° C.

### Example 3—PLGA Stent Reinforced with HA Particles

Referring to FIG. 3, a stent (1) was developed for placement in the trachea. Said stent (1) had a body made of PLGA with a composition of 85% PLA and 15% PGA as polymeric matrix and 2% w/w of particles (11) of Hydroxyapatite (HA) as reinforcement. The final dimensions of said stent (1) were 1.5 cm in diameter and 10 cm in length.

For the preparation of the solution, 2% by weight of Hydroxyapatite (HA) was added with respect to the weight of the PLGA. In order to solubilize the HA, 20 mL of CHCl₃ were taken and the weight percentage of HA was added with respect to the grams of PLGA to be prepared. This solution with the HA nanoparticles is sonicated for 10 minutes without adding the PLGA.

After the homogenization of the solution with HA, portions of 1 g to 5 g of PLGA were added little by little until completing 100 g, between each addition of PLGA it was ensured that the previous addition was completely dissolved to add the next one, in order to guarantee the complete homogenization and dissolution of the PLGA in all the material. In this range of addition, HA gave the stent structure (1) greater mechanical resistance, by increasing the average Young's modulus by more than 38%, hardness by more than 120%, in addition to reducing roughness by more than 30% compared to PLGA without any reinforcement.

After integrating these two components (PLGA-HA), the stent body was constructed (1). This process was carried out using a tube with an internal diameter of 1.6 cm, a solid rod with a diameter of 8 mm and two caps with an opening both for the prepared solution and for the release of the gases evaporating from the solution. This assembly was designed so that the constructed stent (1) had a thickness of 4 mm.

Initially, a release solution was added to the rod and tube to prevent damage to the stent, and both sides of the tube were covered with the rod in the center of the tube and the caps.

Subsequently, the PLGA and HA solution was slowly introduced against gravity, i.e., entering the solution from the bottom to the top of the cylinder. Finally, the solution was allowed to dry for the estimated time and then the plugs, the rod and the tube were slowly removed to avoid damage to the constructed stent (1).

### Example 4—PLA Stent Reinforced with MgO Particles

Referring to FIG. 3, a stent (1) for placement in a vein was developed. Said stent (1) had a body made of PLA polylactic acid as the polymeric matrix and 5% w/w of particles (11) of magnesium oxide (MgO) as reinforcing agent. The final dimensions of said stent (1) were 4 mm in diameter and 1 cm in length.

For the preparation of the solution, 5% by weight of MgO with respect to the weight of PLA was added. In order to solubilize the MgO, 5 mL of CHCl₃ were taken and the percentage by weight of MgO with respect to the grams of PLA to be prepared was added. This solution with the MgO nanoparticles is sonicated for 10 minutes without adding the PLA.

After integrating these two components (PLGA-HA), the stent body (1) was constructed using the dip coating technique.

### Example 5—PLGA Stent Reinforced with HA Particles, Coated with PVA with PCL Nanocapsules with Curcumin

A stent (1) like the one in Example 3 was developed and coated using the electrospinning technique, taking PVA as the coating matrix and polycaprolactone (PCL) nanocapsules containing Curcumin as an antiproliferative therapeutic agent for cancer.

Prior to the stent body coating process (1), PCL nanocapsules with Curcumin were prepared by means of a process based on the nanoprecipitation technique. After obtaining the nanocapsules with Paclitaxel and Curcumin, a 13% PVA solution was prepared and 5% by weight of the nanocapsules were added, which was then used to generate the stent coating (1) using electrospinning.

The equipment used for electrospinning had a syringe pump with which the system feed flow could be varied, a high voltage source operating at 20 kV, which generated a potential difference that allowed the fibers to form continuously, from the tip of the syringe to the manifold. The equipment had a manifold in the form of a circular drum that rotated at 19 rpm and was located at a distance of 15 cm from the tip of the syringe. The polymer solution in a solvent plus the nanocapsules loaded with the therapeutic agent were placed in 10 mL disposable syringes and the feed flow was 0.5 mL/h.

Additionally, the work cabin where the electrospinning was carried out, was brought to a relative humidity below 60% and a temperature of 28° C.

### Example 6—Tests of Mechanical Properties of the Particulate Material

In order to know the impact of the particles (11) on the mechanical properties of the stent (1) material, in one embodiment of the invention the particles (11) mentioned above were evaluated at concentrations between 1 and 5% w/w of the stent and with particle sizes of the order of nanometers.

The changes in the mechanical properties of the material, when each particle was added, were determined by Atomic Force Microscopy (AFM). Mechanical properties include average Young's modulus, hardness (mechanical strength) and roughness. Said parameters can indicate which is the most suitable reinforcing particle for the stent application.

Young's Modulus. In order to know the initial results and have a benchmark, the first material analyzed was unreinforced PLGA polymer, where the average Young's modulus found for the AFM measurements was 2.134±0.233 GPa, wherein unreinforced PLGA is understood to mean the polymer without the addition of any amorphous particle.

The addition of 1% hydroxyapatite increases the average Young's modulus of the material by 14% to 2.432 GPa, however, there is no significant difference between the unreinforced PLGA and this reinforced material. On the other hand, the addition of 2% and 5% hydroxyapatite does present a significant difference with respect to unreinforced PLGA, achieving an increase in the average Young's modulus of 38.5% and 35.8%, respectively. Between the addition of 2% and 5% there is no significant difference between them. Additionally, the maximum average Young's modulus achieved with the addition of hydroxyapatite is achieved with 2% reaching 2.956±0.281 GPa.

On the other hand, the addition of amorphous magnesium phosphate (AMP) particles at 1%, 2% and 5% significantly increases the average Young's modulus of the material by 42%, 59.5% and 38.6% respectively, with respect to the polymer (PLGA) without reinforcement. On the other hand, the addition of 2% of amorphous magnesium phosphate presents a significant difference between the additions of 1% and 5% as well as a significant difference with respect to (PLGA) without reinforcement, achieving the highest value of the average Young's modulus (3.404 GPa) for this nanoparticle.

On the other hand, the addition of amorphous calcium and magnesium phosphate particles at 1%, 2% and 5% significantly increases the average Young's modulus of the material by 27.2%, 42.6% and 36.9%. respectively with respect to the unreinforced PLGA. On the other hand, the addition of 2% of amorphous calcium and magnesium phosphate presents a significant difference with respect to the addition of 1% and the unreinforced PLGA, but not with respect to the addition of 5%.

The addition of magnesium hydroxide particles at 1%, 2% and 5% increases the average Young's modulus of the material by 37.8%, 26.7% and 14.4%, respectively, with respect to the polymer without reinforcement. The unreinforced PLGA did not present a significant difference with respect to the sample reinforced with 5% magnesium hydroxide, but a significant difference with respect to those of 1% and 2%. The highest value of the average Young's modulus is obtained with the addition of 1% (3.019 GPa) and the additions of 2% (2.776 GPa) and 5% (2.442 GPa) decrease the average Young's modulus. This may be due to the difficulty of dispersing these nanoparticles, added to their physicochemical features, since they were the smallest.

The addition of magnesium oxide particles at 1% w/w, 2% w/w and 5% w/w, significantly increases the average Young's modulus of the material by 36.8%, 36.8% and 55.8% respectively with respect to the polymer without reinforcement. In this case, the three levels of evaluated magnesium oxide nanoparticles show a significant difference with respect to the unreinforced PLGA. It can be seen that the highest value of the average Young's modulus is obtained with the addition of 5% (3.326 GPa).

The values obtained for the average Young's modulus are presented below for each of the evaluated stent materials (1). Said materials correspond to PLGA without reinforcement and multiple combinations of PLGA with five types of reinforcement particles (11) in different concentrations:

TABLE 2
Test results by Atomic Force Microscopy measurement
of the average Young's modulus in different possible
stent materials (1).

| | Mean Young's |
|---|---|
| Material | Modulus [Gpa] |
| PLGA without reinforcement | 2.13447 |
| PLGA reinforced with 1% Hydroxyapatite | 2.43222 |
| PLGA reinforced with 5% Magnesium | 2.44211 |
| Hydroxide | |
| PLGA reinforced with 1% Amorphous | 2.71513 |
| Calcium Magnesium Phosphate | |
| PLGA reinforced with 2% Magnesium | 2.77590 |
| Hydroxide | |
| PLGA reinforced with 5% Hydroxyapatite | 2.89950 |
| PLGA reinforced with 1% Magnesium Oxide | 2.92053 |
| PLGA reinforced with 5% Amorphous | 2.92132 |
| Calcium Magnesium Phosphate | |
| PLGA reinforced with 2% Magnesium Oxide | 2.92175 |
| PLGA reinforced with 2% Hydroxyapatite | 2.95608 |
| PLGA reinforced with 5% Amorphous | 2.95850 |
| Magnesium Phosphate | |
| PLGA reinforced with 1% Magnesium | 3.01895 |
| Hydroxide | |
| PLGA reinforced with 1% Amorphous | 3.03065 |
| Magnesium Phosphate | |
| PLGA reinforced with 2% Amorphous | 3.04325 |
| Calcium Magnesium Phosphate | |
| PLGA reinforced with 5% Magnesium Oxide | 3.32684 |
| PLGA reinforced with 2% Amorphous | 3.40425 |
| Magnesium Phosphate | |

Referring to Table 1, the highest average Young's modulus is shown by the material reinforced with 2% amorphous magnesium phosphate followed by 5% magnesium oxide. The acicular morphology of the amorphous magnesium phosphate nanoparticles favors the performance of this material as reinforcement.

Finally, the reinforced materials that show a 40% increase in the average Young's modulus compared to the average Young's modulus of PLGA without reinforcement, i.e., that exceed 3 GPa are: PLGA with 5% magnesium oxide, PLGA reinforced with 1% magnesium hydroxide nanoparticles, PLGA with 1% amorphous magnesium phosphate and PLGA with 2% amorphous calcium magnesium phosphate.

Hardness

On the other hand, the nanoindentation carried out by AFM was used, in the materials of the stent (1) disclosed in Table 1, to carry out an analysis of the average hardness of said materials from the force curves and the force maps registered during said nanoindentation.

The average hardness found for the polymer without any reinforcement was 0.485±0.109 GPa. This value obtained for the average hardness of the unreinforced material is the starting point to compare with the results obtained for the average hardness of the samples reinforced with the five nanoparticles at the three levels.

The addition of 1%, 2%, and 5% hydroxyapatite increases the average hardness of unreinforced PLGA by 3.3%, 129%, and 109%, respectively. The highest hardness value was obtained with 5% hydroxyapatite (4.96 GPa). However, the highest average value was achieved with 2% hydroxyapatite 1.110 GPa.

The addition of 1%, 2% and 5% magnesium phosphate show an increase in the average hardness of PLGA without reinforcement of 67.4%, 117% and 132%, respectively.

The addition of 1%, 2% and 5% of amorphous calcium and magnesium phosphate show an increase in the average hardness of PLGA without reinforcement of 51%, 51.5% and 62.8%, respectively.

The addition of 1%, 2% and 5% of magnesium hydroxide show an increase with respect to the average hardness of PLGA without reinforcement by 50.1%, 45.9% and 41%, respectively. However, as the addition of magnesium hydroxide increases, the average hardness of the material decreases.

The addition of 1%, 2% and 5% magnesium oxide show an increase with respect to the average hardness of PLGA without reinforcement by 131%, 127% and 175%, respectively.

The following are the average hardness values obtained for each of the evaluated stent materials (1). Said materials correspond to PLGA without reinforcement and multiple combinations of PLGA with five types of reinforcement particles (11) in different concentrations:

TABLE 3
Test results by Atomic Force Microscopy measurement of
the average hardness in different possible stent materials (1).

| | Medium |
|---|---|
| | Hardness |
| Material | [GPa] |
| PLGA without reinforcement | 0.485319 |
| PLGA reinforced with 1% Hydroxyapatite | 0.501278 |
| PLGA reinforced with 5% Magnesium Hydroxide | 0.683672 |
| PLGA reinforced with 2% Magnesium Hydroxide | 0.708846 |
| PLGA reinforced with 1% Magnesium Hydroxide | 0.728232 |
| PLGA reinforced with 1% Amorphous Calcium | 0.731333 |
| Magnesium Phosphate | |
| PLGA reinforced with 2% Amorphous Calcium | 0.735500 |
| Magnesium Phosphate | |
| PLGA reinforced with 5% Amorphous Calcium | 0.790079 |
| Magnesium Phosphate | |
| PLGA reinforced with 1% Amorphous Magnesium | 0.812154 |
| Phosphate | |
| PLGA reinforced with 5% Hydroxyapatite | 1.012650 |
| PLGA reinforced with 2% Amorphous Magnesium | 1.051770 |
| Phosphate | |
| PLGA reinforced with 5% Magnesium Oxide | 1.102530 |
| PLGA reinforced with 2% Hydroxyapatite | 1.109920 |
| PLGA reinforced with 1% Magnesium Oxide | 1.121200 |
| PLGA reinforced with 5% Amorphous Magnesium | 1.127430 |
| Phosphate | |
| PLGA reinforced with 5% Magnesium Oxide | 1.336580 |

Referring to Table 2, the highest hardness was shown by the material reinforced with 5% magnesium oxide, followed by 5% amorphous magnesium phosphate. Seven tests were found with an average hardness value greater than 1 GPa, corresponding to materials reinforced with nanoparticles at 2% and 5% hydroxyapatite, 2% and 5% amorphous magnesium phosphate, 1%, 2% and 5% magnesium oxide, respectively.

Roughness Relative to the earlier Young's modulus and hardness tests presented, surface conditions can have significant effects on your results, causing average Young's modulus and hardness values to increase or decrease with extraction depth. For example, when a penetration tip contacts a rough surface, the initial contacts may be rough. If this initial contact is recognized as the “surface” by the test instrument, the hardness and elastic modulus results may be wrong. Therefore, the measurement of the roughness in the measurements of the mechanical properties will be directly proportional to the errors in the measurements, i.e., the greater the roughness, the greater the probability of an error in the measurement of the mechanical properties. Therefore, the surfaces that showed less roughness will provide more reliable values of hardness and Young's modulus, and for this reason a statistical analysis is made in an area of the sample to guarantee and reduce the errors of the test values.

According to the above, roughness samples were made following the ISO 4287 standard norms, using an analysis in a 3 mm line for a time of 15 seconds. Each sample had 3 measurements in different places with a scanning time of 15 seconds. Each sample used had an area of 10 mm². With these measurements we wanted to confirm the influence of the nanoparticles on the surface conditions of the polymer.

In addition, considering that the sample manufacturing system generates two sides, one exposed to the air, which is where the solvent evaporates, and the other exposed to the glass, which is the support or mold in which the test tubes were manufactured, it was characterized on both sides to see if there was a significant difference between both sides. The comparison of the properties on both sides was carried out to rule out the migration of the particles towards any of the sides and thus guarantee a homogeneous behavior of the material.

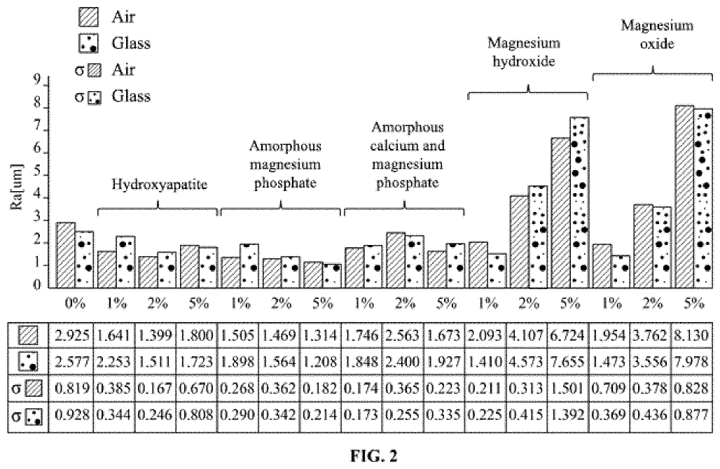

FIG. 2 shows which of the nanoparticles generated a lower roughness when embedded within the PLGA. Nanoparticle additions change or affect the roughness, either decreasing or increasing it depending on the nanoparticle. On the other hand, there is no significant difference between the sides that would indicate sedimentation or migration of the nanoparticles towards any of them. The addition of 1% of the five evaluated nanoparticles decreases the roughness, where the average roughness for the side exposed to air was 1.788 μm and that exposed to glass was 1.776 values lower than those found in the sample without reinforcement of nanoparticles that presented a roughness of 2.925 μm on the side exposed to air and 2.577 μm on the side exposed to glass.

Referring again to FIG. 2 for the specimens reinforced with hydroxyapatite, amorphous magnesium phosphate and amorphous calcium and magnesium phosphate at 2%, the roughness decreased compared to unreinforced PLGA, while for the specimens made with 2% magnesium oxide and hydroxide, the roughness increased. The highest roughness was achieved with the addition of 5% magnesium oxide nanoparticles, while the lowest was with 2% amorphous magnesium phosphate. The average of all the roughness measurements obtained with the nanoparticles for the side exposed to air was 2.792 μm and the average roughness for the side exposed to glass was 2.865 μm. The amorphous magnesium phosphate particles are the only ones that decrease in roughness as the percentage of nanoparticles added increases. Contrary to amorphous magnesium phosphate, magnesium oxide and hydroxide nanoparticles increase roughness as dosage increases.

According to the aforementioned tests related to Young's modulus, hardness and roughness, it is considered that a significant increase in Young's modulus indicates that the material is more rigid, which indicates greater resistance to elastic deformations, i.e., greater capacity to support efforts without acquiring large deformations, which is preferable for the manufacture of the stent (1) of the present disclosure. In addition, it is sought that the added particles serve as a reinforcer that allows a significant improvement of the mechanical properties of the stent material (1) with the least addition, affecting as little as possible other properties of said stent material (1) such as its bioabsorption or biocompatibility.

On the other hand, the hardness of the material, which indicates resistance to scratching or penetration, must also be taken into account when choosing the reinforcing particles. The elongated shapes favor the reinforcement of the materials with respect to other particles with aspect ratios lower or close to one. On the other hand, the more crystalline particles take longer to be bioabsorbed.

Conclusions Based on Evidence

In one embodiment of the stent (1) and according to the above considerations and the results obtained in the AFM tests, it is considered that the particles with the best overall performance as reinforcers are hydroxyapatite (HA) and amorphous magnesium phosphate (AMP).). Particularly, when 2% amorphous magnesium phosphate (AMP) is added to PLGA, the average Young's modulus increases by 59.51% and the hardness of the material is 116.9% higher than PLAG without reinforcement.

In addition, the synthesis process of amorphous magnesium phosphate (AMP) is simpler because it only requires microwave synthesis for 5 minutes, while the synthesis of other particles such as hydroxyapatite requires 30 minutes of microwave irradiation and subsequently requires a calcination process. Similarly, the production of magnesium hydroxide requires a gel formation process that takes at least 12 hours and to convert magnesium hydroxide into magnesium oxide, a calcination process is also required.

On the other hand, the bioabsorbability of amorphous magnesium phosphate (AMP) is better than that of the other particles due to its morphology. Additionally, amorphous magnesium phosphate (AMP) is easily incorporated into PLGA without presenting agglomerations of the reinforcing particles.

### Example 7— Crosslinking Tests

In order to know the effect of crosslinking on the coating, swelling and degradation tests were performed on PVA coatings with glutaraldehyde-ethanol crosslinking and without crosslinking. The non-crosslinked coating swells faster and in less time, it also achieves a higher swelling percentage than the crosslinked coating. Rapid swelling of the non-crosslinked coating was observed, while the crosslinked coatings only hydrated.

The non-crosslinked coating begins to degrade before seven minutes, where the percentage of swelling and degradation begins to decrease to 403%, then 15 minutes to 349% and 30 minutes to 32%. On the other hand, the crosslinked coating presents a swelling and increasing degradation value between time zero and 30 minutes, indicating that the material during this time has not degraded.

The non-crosslinked sample completely degraded by the seventh day, while the crosslinked coating achieves a maximum swelling value of 457% by day 15 and subsequently begins to decrease to 133% by day 30, associated with the fact that the coating begins its degradation process.

The coating releases the drug through two phenomena, an initial one associated with diffusion while the system has not begun to degrade and another associated with erosion or degradation of the system, which can be verified with the release test which will allow identifying the contribution of each of these phenomena to drug release.

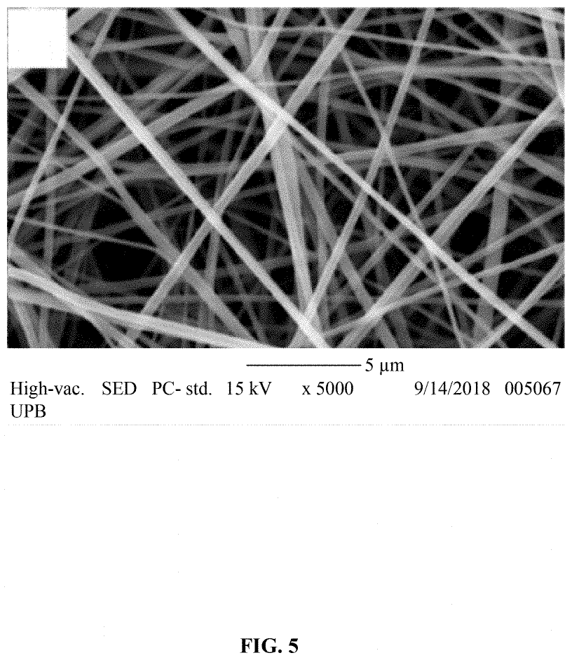

Referring to FIG. 5, a micrograph taken with the scanning electron microscope technique (Scanning Electron Microscope, SEM) at 5000× magnification of the coating modality consisting of a 13% w/v PVA matrix with Paclitaxel-loaded PLGA nanocapsules.

In FIG. 5, it is identified that the fibers obtained do not present beads or points where the fiber widens and lodges a high concentration of nanoparticles, which is not desirable if a more uniform distribution of the nanocapsules is sought and therefore of the therapeutic agent inside the fibers. The fibers observed in FIG. 5 have a suitable morphology for covering the stent (1). In this example, the use of Poloxamer 407 as a surfactant in the solution containing nanocapsules helps to reduce the surface tension of said PVA solution to be electrospun. This decreases the appearance of beads or thickening points in the electrospun fibers of the final coating.

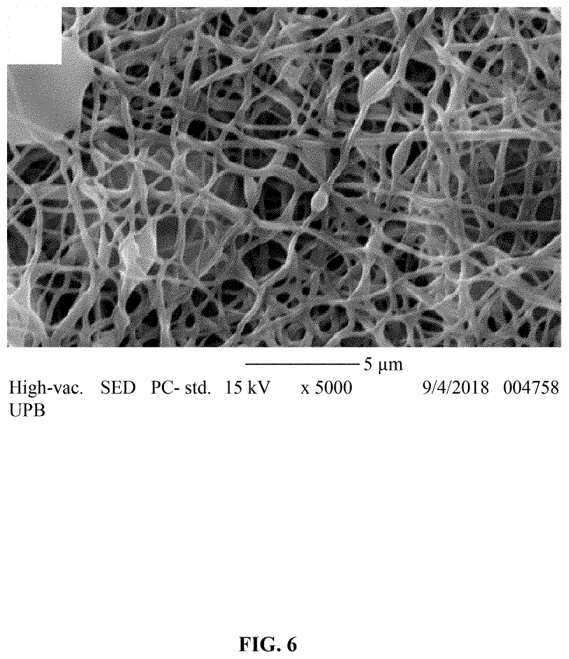

Referring to FIG. 6 and in an example of the invention, PVA fibers at 13% w/v with paclitaxel-loaded PLGA nanocapsules that are crosslinked by means of 25% glutaraldehyde are observed.

In one embodiment of the stent (1), the coating material can be a PVA filament with a diameter between 100 nm and 500 nm.

1. A stent comprising:
a material selected from a biocompatible material, a bioabsorbable material, and combinations thereof; and
particles selected from biocompatible amorphous particles, bioabsorbable amorphous particles and combinations thereof.

2. The stent of claim 1, further having a coating comprising:
a material selected from a biocompatible material, a bioabsorbable material, and combinations thereof;
nanocapsules; and
a therapeutic agent encapsulated in the nanocapsules.

3. The stent according to any of claims 1 and 2, wherein the stent material is selected from the group including glycolic acid, lactic acid, lactic-co-glycolic acid, polylactic-co-glycolic acid, polycaprolactone, chitosan and combinations thereof.

4. The stent according to any of claims 1 to 3, wherein the amorphous particles are selected from the group of amorphous magnesium phosphate, amorphous calcium magnesium phosphate, hydroxyapatite, magnesium hydroxide, magnesium oxide and mixtures thereof.

5. The stent according to any of the preceding claims, wherein the stent material is polylactic-co-glycolic acid and the particle material is amorphous magnesium phosphate.

6. The stent according to any of claims 2 to 5, wherein the coating material is selected from the group of cisplatin, collagen, polyvinyl alcohol (PVA), polylactic acid (PLA), polyglycolic acid (PGA), polylactic acid-co-glycolic (PLGA), and mixtures thereof.

7. The stent according to any of claims 2 to 6, wherein the nanocapsules are selected from the group of glycolic acid, lactic acid, lactic-co-glycolic acid, polylactic-co-glycolic acid, polycaprolactone, chitosan and mixtures thereof.

8. The stent according to any of claims 2 to 7, where the coating material is polyvinyl alcohol and the material of the nanocapsules is co-glycolic acid.

9. The stent according to any of claims 1 to 8, wherein the representative sphere diameter of the particles is between 1 nm and 1 μm.

10. The stent according to any of claims 2 to 9, wherein the coating material is a polyvinyl alcohol filament with a diameter of 100 nm to 500 nm.

11. The stent according to any of claims 2 to 10, wherein the therapeutic agent is selected from the group including antiproliferative agents, antithrombotic agents, anticoagulant agents, anti-inflammatory agents, antineoplastic agents, antiplatelet agents, antifibrosis agents, antimitotic agents, antibiotics, antiallergic agents, antioxidant agents, chemotherapeutic agents, cytostatic agents, cell migration inhibitors, immunosuppressants, and combinations thereof.

12. The stent according to any of claims 2 to 11, wherein the therapeutic agent is selected from the group including Mitomycin, Vincristine, Paclitaxel and Curcumin.

13. The stent of claim 12, where the therapeutic agent is an antiproliferative agent selected from Paclitaxel, Curcumin and combinations thereof.

14. The stent according to any of claims 2 to 13, wherein the coating has a therapeutic agent concentration of 50 μg/cm² to 150 μg/cm².
